(19) United States
(12) Patent Application Publication
(10) Pub. No.: US 2010/0247693 A1
(43) Pub. Date: Sep. 30, 2010

(54) COSMETIC FORMULATION TO TREAT ROSACEA TELANGIECTASIA

(76) Inventor: Jan L. Marini, San Jose, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/410,336

(22) Filed: Mar. 24, 2009

(51) Int. Cl. A61K 36/185; A61K 38/02; A61P 17/00

(52) U.S. Cl. 424/769; 514/2

(57) ABSTRACT

The present invention features novel cosmetic skin care compositions for improving the appearance of rosacea-affected skin.

### BACKGROUND OF THE INVENTION

Rosacea is a common but often misunderstood condition that is estimated to affect over 45 million people worldwide. It begins as erythema (flushing and redness) on the central face and across the cheeks, nose, or forehead but can also less commonly affect the neck and chest. As rosacea progresses, other symptoms can develop such as semi-permanent erythema, telangiectasia (dilation of superficial blood vessels on the face), red domed papules (small bumps) and pustules, red gritty eyes, burning and stinging sensations. Rosacea sufferers often report periods of depression stemming from cosmetic disfigurement, painful burning sensations, and decreases in quality of life.

There are several rosacea subtypes, including erythematotelangiectatic rosacea and papulopustular rosacea, which is characterized by permanent redness with a tendency to flush easily. It is also common to have small blood vessels visible near the surface of the skin (telangiectasias) and possibly burning or itching sensations.

Rosacea affects both sexes, but is almost three times more common in women, and has a peak age of onset between 30 and 50. There is an apparent hereditary component and those that are fair-skinned of Celtic and other European ancestries have a higher genetic predisposition to developing it.

Triggers that cause episodes of flushing and blushing play a part in the development of rosacea. Exposure to temperature extremes can cause the face to become flushed as well as strenuous exercise, heat from sunlight, severe sunburn, stress, anxiety, cold wind, moving to a warm or hot environment from a cold one such as heated shops and offices during the winter. There are also some foods and drinks that can trigger flushing, these include alcohol, foods and beverages containing caffeine (especially, hot tea and coffee), foods high in histamines and capsaicins.

Some acne and wrinkle treatments have been reported to worsen rosacea, including microdermabrasion, chemical peels, and high dosages of tretinoin. Steroid use can also aggravate rosacea.

Current methods for treating rosacea varies from patient to patient depending on severity and subtypes. A subtype-directed approach to treating rosacea patients is recommended to dermatologists. It is important to have a therapeutic skin care regimen including protection from the sun. Dermatological vascular or intense pulsed light lasers have been used, where light penetrates the epidermis and heats capillaries of the dermal layer.

There is a continuing need for cosmetic formulations to address the cosmetic issues associated with rosacea subtypes, including telangiectasic rosacea. The compositions and their methods of use presented herein meet that need.

### SUMMARY OF THE INVENTION

The present invention features the redness associated with rosacea, e.g. in rosacea telangiectasia, and papulopustular rosacea. Specific blemishes include rosacea lesions, papules and pustules. Specifically, the skin care compositions presented herein contain a synergistic combination of: azaleic acid; a retinoid, e.g. retinol; one or more vitamin K compounds; and peptides, for use in improving the appearance of rosacea-affected regions of the skin. The formulation is provided in a cosmetically acceptable vehicle(s), which may further comprise skin soothing/conditioning agents, and permeation enhancers, e.g. transcutol. Accordingly, the synergistic combinations of the active ingredients of the invention are formulated as skin care cosmetic compositions that can be applied directly to the skin so as to improve the appearance of skin texture and color. The compositions may additionally provide cosmetic benefit for acne, discoloration, oily skin, aging skin, spider veins, and sun damage.

According to the first aspect of the invention, there is provided a synergistic cosmetic composition comprising: i) 0.05-10% by weight azaleic acid; ii) 0.001-2% by weight a retinoid; iii) 0.01-5% by weight of one or more vitamin K compounds; and iv) 10 PPM-500 PPM peptides. In one embodiment of the invention, the retinoid is retinol. In some embodiments the composition further comprises from 0.5% up to 10% resveratrol. In some embodiments the composition further comprises from 0.5% up to 10% pomegranate extract.

In the second aspect of the invention, a method is provided for improving the appearance of rosacea-affected regions of the skin, the method comprising applying topically a cosmetic formulation with a synergistic cosmetic composition comprising: i) 0.05-10% by weight azaleic acid; ii) 0.001-2% by weight a retinoid; iii) 0.01-5% by weight of one or more vitamin K compounds; and iv) 10 PPM-500 PPM peptides. The composition is topically administered as a lotion or cream for a period of time sufficient to accomplish the desired effect. In some embodiments the composition is administered once daily, or twice daily, and for at least about one week, at least about two weeks, at least about one month, or longer as desired.

### DETAILED DESCRIPTION OF THE EMBODIMENTS

Topical compositions are provided for improving the appearance of the redness associated with rosacea, e.g. in rosacea telangiectasia, and papulopustular rosacea. Specific blemishes include rosacea lesions, papules and pustules. The compositions may additionally provide cosmetic benefit for acne, discoloration, oily skin, aging skin, spider veins, and sun damage.

Rosacea is a chronic inflammatory disorder characterized by facial flushing, telangiectasias, erythema, papules, pustules, and in severe cases, rhinophyma. Diagnosis is based on characteristic appearance. Prior methods of treatment depends on severity and include topical metronidazole, topical and oral antibiotics, and laser therapy.

The etiology of rosacea is unknown. Rosacea manifests in 4 phases and is usually limited to the face and scalp. The phases of rosacea are not always sequential. Some patients go directly into the inflammatory stage bypassing the earlier stages. Treatment may cause a patient to return to an earlier stage.

In the “pre-rosacea” phase, patients describe embarrassing flushing and blushing, often accompanied by uncomfortable stinging. Common reported triggers for these flares include sun exposure, emotional stress, cold or hot weather, alcohol, spicy foods, exercise, wind, cosmetics, and hot baths or hot drinks. These symptoms persist throughout other phases of the disorder. In the vascular phase, patients develop facial erythema and edema with multiple telangiectasias, possibly as a result of persistent vasomotor instability.

An inflammatory phase often follows, in which sterile papules and pustules (leading to the designation of rosacea as “adult acne”) develop. Some patients go on to develop late-stage rosacea, characterized by coarse tissue hyperplasia of the cheeks and nose (rhinophyma) caused by tissue inflammation, collagen deposition, and sebaceous gland hyperplasia.

### Components of the Cosmetic Compositions

Vitamin K is a group of lipophilic, hydrophobic vitamins that are needed for the posttranslational modification of certain proteins, mostly required for blood coagulation. Chemically they are 2-methyl-1,4-naphthoquinone derivatives. All members of the vitamin K group of vitamins share a methylated naphthoquinone ring structure, and vary in the aliphatic side chain attached at the 3-position. Phylloquinone (also known as vitamin K1, shown below) invariably contains in its side chain four isoprenoid residues, one of which is unsaturated. Vitamin K2 (menaquinone, menatetrenone) has side chains composed of a variable number of unsaturated isoprenoid residues; as shown below, where, where n specifies the number of isoprenoids.

It is generally accepted that the naphthoquinone is the functional group, so that the mechanism of action is similar for all K-vitamins.

### Vitamin K1, Phylloquinone

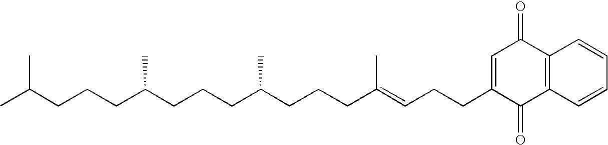

### Vitamin K2, Menaquinone

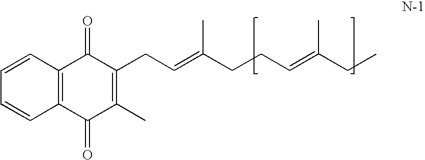

The cosmetic compositions of the present invention may comprise any one of the vitamin K compounds, as described herein, or may comprise any combination of the same, as there is a common mechanism of action. The cosmetic compositions of the present invention may contain vitamin K in amounts that are safe and effective, for instance, at concentrations of at least about 0.01%, at least about 0.1%, around about 1%, at and usually not more than about 5% (weight/weight).

Azelaic acid is a saturated dicarboxylic acid that is produced by Malassezia furfur, a yeast that lives on normal skin. When applied topically to the skin in a combination formulation it acts synergistically with the other agents of the of the invention. The cosmetic compositions of the present invention may contain azaleic acid in amounts that are safe and effective, for instance, at concentrations of at least about 0.05%, at least about 0.5%, around about 2%, around about 5%, and usually not more than about 10% (weight/weight).

Retinoid. Retinol (vitamin A), is a fat-soluble vitamin. It belongs to the family of chemical compounds known as retinoids. Derivatives include retinal, also known as retinaldehyde, which can be reversibly reduced to produce retinol or it can be irreversibly oxidized to produce retinoic acid (tretinoin, isotretinoin). The best described active retinoid metabolites are 11-cis-retinal and the all-trans and 9-cis-isomers of retinoic acid. Retinol may be measured in international units (IU), where 1 IU of retinol is equivalent to approximately 0.3 micrograms (300 nanograms).

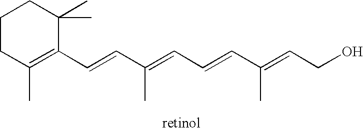

Cosmetic formulation of the present invention comprise at least about 0.001% by weight of a retinoid, usually at least about 0.01%, more usually at least about 0.1%, and from about 0.5% to about 2%, e.g. about 1%. A preferred compound for the compositions of the present invention is retinol.

Peptides, including but not limited to, di-, tri-, tetra-, and pentapeptides, as well as oligo-peptides of from about 6 to about 30 amino acids in length and derivatives thereof, are included as cosmetic benefit agents of the present invention in amounts that are safe and effective. Such peptides are usually acylated, comprising at least one lipid moiety, which moiety may be myristoyl, palmitoyl, etc., which increases the hydrophobicity of the peptide. Myristoyl or palmitoyl pentapeptides are of particular interest, as is the use of thymosin β4. As used herein, “peptides” refers to both naturally occurring peptides and synthesized peptides. Below is a non-limiting list of exemplary peptide agents that find use in the cosmetic compositions of the present invention.

Certain peptide agents of interest stimulate the macromolecules of the dermis. For example, synthetic peptides such as lamin, biopeptide CL or palmitoyloligopeptide (SEDERMA) activate the synthesis of collagen. In addition, natural peptides extracted from plants, such as the soya bean hydrolyzate marketed by the company COLETICA under the trademark Phytokine™, also provide this activity.

Certain other peptide agents act on the synthesis of fibronectin, such as the palmitoyl pentapeptide marketed by the company SEDERMA under the trademark Matrixil™. Still other of these peptides inhibit metalloproteinases, such as oligopeptides and lipopeptides, lipoamino acids, and malt extract marketed by the company COLETICA under the trademark Collalift™, while some peptide agents inhibit serine proteases, such as leukocyte elastase or cathepsin G, including the peptide extract of seeds of leguminous plants (Pisum sativum) which is marketed by the company LSN under the trademark Parelastyl™ and certain pseudo-dipeptides.

Some peptide agents that find use in the cosmetic compositions of the present invention stimulate the proliferation of fibroblasts, including plant polypeptides (or extracts) from soya bean (e.g., Eleseryl SH-VEG 8™ marketed by the company LSN, or Raffermine™ marketed by the company SILAB).

In addition to the activities noted above, certain peptide agents may provide anti-bacterial or anti-fungal activity to the cosmetic compositions of the invention. For example, see U.S. Pat. No. 6,835,536, which describes numerous cationic antimicrobial peptides (e.g. indolicidins or analogs or derivatives thereof derived from natural sources or produced synthetically) that find use in topically-applied compositions for the treatment of acne. Similarly, U.S. Pat. No. 6,713,078 described granulysin peptides useful in the topical treatment of acne.

The peptide agents of the present invention are formulated at an effective concentration within the subject cosmetic compositions, meaning at a concentration that provides the intended benefit when applied topically. An effective concentration of peptide or peptide-like compounds is preferably in a range of at least about 10 parts per million (PPM), more usually at least about 50 PPM, at least about 100 PPM, and less than about 500 PPM, usually less than about 250 PPM.

In addition to the combination of azaleic acid, retinoid, vitamin K and peptides, the cosmetic formulation may include other active agents. In some embodiments, the formulation comprises permeation enhancer, e.g. transcutol, (diethylene glycol monoethyl ether), which may be provided at a weight/weight concentration of from about 0.1% to about 10%, usually from about 2.5% to about 7.5%, more usually about 5%.

Resveratrol. The compositions of the invention optionally include up to 1%, up to 5%, up to 10% resveratrol (trans-resveratrol). Resveratrol (3,5,4′-trihydroxystilbene) is a polyphenolic phytoalexin. It is a stilbenoid, a derivate of stilbene, and is produced in plants. It exists as two geometric isomers: cis-(Z) and trans-(E). The trans-form can undergo isomerisation to the cis- form when exposed to ultraviolet irradiation. In grapes, resveratrol is found primarily in the skin, and in muscadine grapes, also in the seeds. Resveratrol supplements are often derived from Japanese knotweed, which contains up to 187 mg/kg in the dried root and can be concentrated in an extract up to 50%.

Pomegranate extract. The compositions of the invention optionally include up to 1%, up to 5%, up to 10% pomegranate extract. Pomegranate (Punica granatum L.) juice and pomegranate extracts exhibit potent biological properties attributable to the presence of polyphenols known as ellagitannins. These hydrolysable tannins are present in high levels in pomegranates and include punicalagin anomers commonly referred to as punicalagins, punicalin, gallagic acid and ellagic acid. Pomegranate ellagitannins have been identified as the active antioxidant compounds responsible for protecting low-density lipoprotein cholesterol from oxidation in vivo, a key step in the pathogenesis of atherosclerosis. The punicalagins are key compounds responsible for the antioxidant properties of pomegranate extracts.

Pomegranate extract has demonstrated a variety of beneficial functions including antioxidant and anti-viral activity. Ellagic acid effectively protects cells from damaging free radicals. Additional phenolic compounds found in pomegranate known as anthocynadins (also well known scavengers of free radicals) combine synergistically with Ellagic acid to greatly augment pomegranate's potency as an antioxidant.

### Skin Soothing/Conditioning Agents

The cosmetic compositions of the present invention may contain agents that sooth, condition and/or heal the skin and hair. One such agent is panthenol, a pro-vitamin moisturizing agent related to Vitamin E. Panthenol is easily incorporated into cosmetic formulations and readily penetrates the skin. Panthenol derivatives (e.g., ethyl panthenol) also find use in the compositions of the invention as do agents such as aloe vera, pantothenic acid and its derivatives, allantoin, bisabolol, and dipotassium glycyrrhizinate. One or more of these agents may be provided in an amount from about 1 to about 25% by weight. Many other skin conditioning/soothing agents can be included in the subject compositions, some of which are discussed below.

The compositions of the invention may optionally comprise other skin benefit materials. These include estradiol; progesterone; pregnanalone; coenzyme Q10; methylsolanomethane (MSM); copper peptide (copper extract); plankton extract (phytosome); kojic acid; ascorbyl palmitate; all-trans-retinol; broparoestrol; estrone; adrostenedione; androstanediols; etc. The steroids will generally be present at a concentration of less than about 5% or about 10% of the total by weight of the composition, while the other skin benefit materials may be present at higher levels, for example as much as about 10 to about 15%.

The compositions may further comprise sunscreens to lower skin's exposure to harmful UV rays. Sunscreens include those materials commonly employed to block ultraviolet light. Illustrative compounds are the derivatives of PABA, cinnamate and derivatives of salicylate (other than ferulyl salicylate). For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. Dermascreen may also be used. The exact amount of sunscreen employed in the compositions can vary depending upon the degree of protection desired from the sun's UV radiation.

The amounts of cosmetic or dermatological auxiliaries and additives and perfume to be used in each case can easily be determined by simple exploratory experiments by the person skilled in the art, depending on the nature of the product in question.

### Cosmetically Acceptable Vehicle

The compositions of the invention include a cosmetically acceptable vehicle to act as a diluent, dispersant or carrier for the active agents, so as to facilitate distribution and uptake when the composition is applied to the skin. Vehicles other than or in addition to water can include liquid or solid emollients, solvents, humectants, thickeners and powders.

The cosmetically acceptable vehicle will usually form 5% to 99.9%, preferably from 25% to 80% by weight of the composition, and can, in the absence of other cosmetic adjuncts, form the balance of the composition.

The compositions may be in the form of aqueous, aqueous/alcoholic or oily solutions; dispersions of the lotion or serum type; anhydrous or lipophilic gels; emulsions of liquid or semi-liquid consistency, which are obtained by dispersion of a fatty phase in an aqueous phase (O/W) or conversely (W/O); or suspensions or emulsions of smooth, semi-solid or solid consistency of the cream or gel type. These compositions are formulated according to the usual techniques as are well known to this art.

When the compositions of the invention are formulated as an emulsion, the proportion of the fatty phase may range from 5% to 80% by weight, and preferably from 5% to 50% by weight, relative to the total weight of the composition. Oils, emulsifiers and co-emulsifiers incorporated in the composition in emulsion form are selected from among those used conventionally in the cosmetic or dermatological field. The emulsifier and co emulsifier may be present in the composition at a proportion ranging from 0.3% to 30% by weight, and preferably from 0.5% to 20% by weight, relative to the total weight of the composition.

When the compositions of the invention are formulated as an oily solution or gel, the fatty phase may constitute more than 90% of the total weight of the composition.

The compositions of the invention may be in the form of body cleansing compositions. As such, these compositions may contain at least one wash-active surfactant in an aqueous base are preferred embodiments of the invention. The surfactants can be present, alone or in a mixture, and are contained in an amount of preferable from 1 to 50% by weight, especially preferably from 1 to 30% by weight. Nonionic surfactants, amphoteric surfactants, zwitterionic surfactants and anionic surfactants are generally suitable.

Suitable anionic surfactants include, e.g. alkaline or alkaline earth salts, alpha-olefin sulfonates, sulfosuccinates, disodium laureth-3 sulfosuccinate, disodium PEG-5 lauryl citrate sulfosuccinate, disodium ricinolamido MEA-sulfosuccinate or disodium laurylamido MEA-sulfosuccinate and alkyl ether carboxylates.

Suitable nonionic surfactants include e.g. alkoxylated fatty alcohols, alkoxylated fatty acid esters, alkoxylated partial glycerides, saturated or unsaturated fatty acids, alkoxylated polyol esters, and alkylpolyglucosides, such as coconut glucosides, lauryl glycosides or decylglucosides. For example, ethoxylated lauryl alcohol, tetradecyl alcohol, cetyl alcohol, oleyl alcohol or stearyl alcohol, which are used alone or in mixtures with each other, as well as fatty alcohols of ethoxylated lanolin, are suitable as fatty alcohol ethoxylates. Furthermore the ethoxylated fatty acid sugar esters known as nonionic surfactants, especially ethoxylated sorbitan fatty acid ester, are suitable for use in the cosmetic preparations according to the invention. The suitable ethoxylated fatty acid sugar esters include those marketed under the trade names Tween™ and Arlacel™ by ICI surfactants and the alkyl-polyglycosides, which are marketed under the trade names Plantaren™ or Plantacare™ by Henkel or under the trade name Oramix™ by Seppic.

Suitable amphoteric surfactants include for example betaines, such as cocoamidopropylbetaine or lauryl betaine, sulfobetaines, such as cocoamidopropyl hydroxysultaine, glycinates, such as cocoamphoglycinate (INCI-name: sodium cocoamphoacetate) and diglycinates and propionates, such as cocoampho-propionate.

The compositions of the invention may also contain additives and adjuvants which are conventional in the cosmetic, pharmaceutical or dermatological field, such as hydrophilic or lipophilic gelling agents, hydrophilic or lipophilic active agents, preservatives, antioxidants, solvents, fragrances, fillers, bactericides, odor absorbers and dyestuffs or colorants. The amounts of these various additives and adjuvants are those conventionally used in the field, and, for example, range from 0.01% to 10% of the total weight of the composition. Depending on their nature, these additives and adjuvants may be introduced into the fatty phase or into the aqueous phase.

Exemplary oils which may be used according to this invention include mineral oils (liquid petrolatum), plant oils (liquid fraction of karite butter, sunflower oil), animal oils (perhydrosqualen(e), synthetic oils (purcellin oil), silicone oils (cyclomethicone) and fluoro oils (perfluoropolyethers). Fatty alcohols, fatty acids (stearic acid) and waxes (paraffin wax, carnauba wax and beeswax) may also be used as fats.

Emulsifiers which may be used include glyceryl stearate, polysorbate 60, PEG-6/PEG-32/glycol stearate mixture, etc. Solvents which may be used include the lower alcohols, in particular ethanol and isopropanol, and propylene glycol.

Hydrophilic gelling agents include carboxyvinyl polymers (carbomer), acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose and polyethylene.

An oil or oily material may be present, together with an emollient to provide either a water-in- oil emulsion or an oil-in-water emulsion, depending largely on the average hydrophilic-lipophilic balance (HLB) of the emollient employed. Levels of such emollients may range from about 0.5% to about 50%, preferably between about 5% and 30% by weight of the total composition. Emollients may be classified under such general chemical categories as esters, fatty acids and alcohols, polyols and hydrocarbons.

Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eurcate and stearyl oleate. Preferred esters include coco-caprylate/caprate (a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids.

Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbons which may serve as emollients are those having hydrocarbon chains anywhere from 12 to 30 carbon atoms. Specific examples include mineral oil, petroleum jelly, squalene and isoparaffins.

Another category of functional ingredients within the cosmetic compositions of the present invention are thickeners. A thickener will usually be present in amounts anywhere from 0.1 to 20% by weight, preferably from about 0.5% to 10% by weight of the composition. Exemplary thickeners are cross-linked polyacrylate materials available under the trademark Carbopol. Gums may be employed such as xanthan, carrageenan, gelatin, karaya, pectin and locust beans gum. Under certain circumstances the thickening function may be accomplished by a material also serving as a silicone or emollient. For instance, silicone gums in excess of 10 centistokes and esters such as glycerol stearate have dual functionality.

Powders may be incorporated into the cosmetic composition of the invention. These powders include chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof.

Other adjunct minor components may also be incorporated into the cosmetic compositions. These ingredients may include coloring agents, opacifiers and perfumes. Amounts of these other adjunct minor components may range anywhere from 0.001% up to 20% by weight of the composition.

Accordingly, a composition of the invention comprises a retinoid, a stable kojic acid derivative, and a resorcinol derivative, which may be a synergistic combination, and optionally in combination with one or more of a permeation enhancer, an azelaic acid or a derivative thereof, salicylic acid or a derivative thereof, glycolic acid or a derivative thereof, licorice extract, and green tea extract, and/or a cosmetically acceptable vehicle. Furthermore, a composition of the invention may include additional agents or additives that are not in themselves active agents but play a role in promoting the usefulness or effectiveness of an active agent.

Compositions of the invention may be applied to any subject and used to treat a variety of conditions, for example skin redness associated with rosacea, e.g. in rosacea telangiectasia, and papulopustular rosacea. Specific blemishes include rosacea lesions, papules and pustules. The compositions may additionally provide cosmetic benefit for acne, discoloration, oily skin, aging skin, spider veins, and sun damage. A typical composition of the invention is formulated as a solution, lotion, cream, gel, ointment, liniment, solvent, emulsion, dispersion, hydrodispersion, aerosol, propellant, soap, exfoliant or transdermal patch, which may be applied topically to the skin so as to treat, prevent, wash, condition or otherwise effect a condition of the skin.

### Product Use, Form, and Packaging

In use, a quantity of the composition, for example from 1 to 100 ml, is applied to a site of interest from a suitable container or applicator and, if necessary, it is then spread over and/or rubbed into the site using the hand or fingers or a suitable device. The product may be specifically formulated for use as a treatment for a specific area, e.g. the hands, the face, the arms, etc.

The cosmetic composition of the invention can be formulated in any form suitable for application to the site of interest, including a lotion, cream, gel, or the like. The composition can be packaged in any suitable container to suit its viscosity and intended use by the consumer. For example, a lotion or cream can be packaged in a bottle, or a propellant-driven aerosol device or a container fitted with a pump suitable for finger operation. When the composition is a cream, it can simply be stored in a non-deformable bottle or squeeze container, such as a tube or a lidded jar. The invention accordingly also provides a closed container containing a cosmetically acceptable composition as herein defined.

The following examples are put forth so as to provide those of ordinary skill in the art with a complete disclosure and description of how to make and use the subject invention, and are not intended to limit the scope of what is regarded as the invention. Efforts have been made to insure accuracy with respect to the numbers used (e.g. amounts, temperature, concentrations, etc.) but some experimental errors and deviations should be allowed for. Unless otherwise indicated, parts are parts by weight, molecular weight is weight average molecular weight, temperature is in degrees centigrade, and pressure is at or near atmospheric.

### EXAMPLE 1

Example 1 illustrates topical compositions according to the present invention. The compositions can be processed in conventional manner. They are suitable for cosmetic use. In particular the compositions are suitable for application to a site of interest for the treatment of a variety of skin conditions or disorders.

ROSACEA LOTION

| RM NAME | %(w/w) |
|---|---|
| D.I.WATER | q.s. to 100% |
| VITAMIN K | 1.00 |
| AZALEIC ACID | 5.00 |
| RETINOL 10S (0.2% ON ACTIVE BASIS) | 2.00 |
| PEPTIDES | 100 PPM |
| TRANSCUTOL | 5.00 |
| BUTYLENE GLYCOL | 4.00 |
| STEARYL ALCOHOL | 3.00 |
| C12-C15 ALKYL BENZOATE | 3.00 |
| CAPRIC/CAPRYLIC TRIGLYCERIDE | 2.00 |
| CETYL ALCOHOL | 2.00 |
| MODIFIED POTATO STARCH | 2.00 |
| POMEGRANATE EXTRACT (0.05-10%) | 1.00 |
| Red Tea Extract | .05 |
| resveratrol 5% | 5.00 |
| grape seed extract | 1.00 |
| GLYCERIN | 1.00 |
| DIMETHICONE | 1.00 |
| PROPYLENE GLYCOL, DIAZOLIDINYL UREA, | 1.00 |
| METHYLPARABEN, PROPYLPARABEN | |
| GLYCERIN, PROPYLENE GLYCOL, GLYCERYL | 1.00 |
| ACRYLATE/ACRYLIC ACID COPOLYMER | |
| GLYCERIN, PROPYLENE GLYCOL, CUCUMIS | 1.00 |
| SATIVUS (CUCUMBER) FRUIT EXTRACT, | |
| ARNICA MONTANA FLOWER EXTRACT, | |
| ANACYCLUS PYRETHRUM ROOT EXTRACT | |
| HYDROXYETHYLCELLULOSE (AND) SODIUM | 0.80 |
| ACETATE (AND) WATER | |
| EMULSIFYING WAX | 0.50 |
| POLYSORBATE 60 | 0.50 |
| LINSEED OIL, PALM OIL AMINOPROPANEDIOL | 0.10 |
| ESTERS | |
| JAPANESE GREEN TEA EXTRACT | 0.10 |
| DL-ALPHA TOCOPHERYL ACETATE | 0.10 |
| DIPOTASSIUM GLYCYRRHIZATE | 0.01 |
| TOTAL | 100.00% |

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A cosmetic composition for topical application comprising a synergistic combination of:
i) 0.05-10% by weight azaleic acid;
ii) 0.001-2% by weight retinol;
iii) 0.01-5% by weight of one or more vitamin K compounds;
iv) 10 PPM-500 PPM acylated di-, tri-, tetra-, and pentapeptides; and
a cosmetically acceptable vehicle.

2. (canceled)

3. The composition of claim 1, further comprising a permeation enhancer at a concentration of 2.5% to 7.5%.

4. The composition of claim 3, wherein the permeation enhancer is diethylene glycol monoethyl ether.

5. The composition of claim 1, further comprising from 0.5% to 10% resveratrol.

6. A cosmetic composition for topical application comprising a synergistic combination of:
i) 0.05-10% by weight azaleic acid;
ii) 0.001-2% by weight retinol;
iii) 0.01-5% by weight of one or more vitamin K compounds;
iv) 10 PPM-500 PPM acylated di-, tri-, tetra-, and pentapeptides;
from 0.5% to 10% pomegranate extract;
from 0.5% to 10% resveratrol; and
a cosmetically acceptable vehicle.

7. A method of improving the appearance of rosacea-affected skin, comprising:
topically applying a cosmetic composition comprising:
i) 0.05-10% by weight azaleic acid;
ii) 0.001-2% by weight retinoid;
iii) 0.01-5% by weight of one or more vitamin K compounds;
iv) 10 PPM-500 PPM peptides; and
a cosmetically acceptable vehicle.

8. The method of claim 7, wherein the cosmetic composition further comprises a permeation enhancer at a concentration of 2.5% to 7.5%.

9. The method of claim 7, wherein the retinoid is retinol.

10. The method of claim 7, further comprising a permeation enhancer at a concentration of 2.5% to 7.5%.

11. The method of claim 10, wherein the permeation enhancer is diethylene glycol monoethyl ether.

12. The method of claim 7, wherein the cosmetic composition further comprises from 0.5% to 10% resveratrol.

13. The method of claim 7, wherein the cosmetic composition further comprises from 0.5% to 10% pomegranate extract.

14. The method of claim 7, wherein the skin condition is selected from the group consisting of rosacea telangiectasia, and papulopustular rosacea.

15. The method of claim 7, wherein the rosacea-affected skin includes one or more of rosacea lesions, papules and pustules.

16. The method of claim 7, wherein the method additionally provides cosmetic benefit for one or more of acne, discoloration, oily skin, aging skin, spider veins, and sun damage.
